(19) United States
(12) Patent Application Publication
(10) Pub. No.: US 2010/0255084 A1
(43) Pub. Date: Oct. 7, 2010

(54) Medicinal melting capsules for oral mucosal absorption

(76) Inventor: Yoel Ovil, Johannesburg (ZA)

Correspondence Address: Dara L. Onofrio, Esq.;c/o ONOFRIO LAW, Suite 225, 15 N. Mill Street, Nyack, NY, 10960, US

(21) Appl. No.: 12/798,489

(22) Filed: Apr. 5, 2010

Related U.S. Application Data
(60) Provisional application No. 61/212,051, filed on Apr. 6, 2009.

(51) Int. Cl. A61K 9/48; A61P 43/00

(52) U.S. Cl. 424/452

(57) ABSTRACT

A medicinal melting capsule comprised of a material that has the same melting point as the human body temperature and includes a therapeutic amount of medication for use in treatment of a desired medical condition; wherein the medication has been modified for oral mucosal absorption; and a related method for delivering medication to an individual via oral mucosal absorption comprising the steps of providing a melting capsule comprised of a material, that has the same melting point as the human body temperature, and includes a therapeutic amount of medication for use in treatment of a desired medical condition; modifying said medication by manipulating the solvent, pH, buffer material and/or polarity to adjust for oral mucosal absorption; administering the capsule sublingually in the patient such that the capsule melts at the individual's body temperature and delivers the medication directly into the bloodstream of the individual quickly and without gastric complications.

This application claims the benefit of U.S. provisional application No. 61/212,051 filed Apr. 6, 2009, which is incorporated herein in its entirety by reference.

### FIELD OF THE INVENTION

The present invention relates to a medicinal melting capsule designed to deliver modified medications that are absorbed through oral mucous membranes, preferably in the mouth of the individual. In particular, the capsule is made of a material that contains the desired medication in liquid or powder form and melts at body temperature. When the capsule is placed under the tongue of an individual the medication is delivered quickly into the blood stream without gastric complications.

### BACKGROUND OF THE INVENTION

A conventional mode of delivery of active pharmaceutical ingredients is the solid dosage route where the tablet is designed to disintegrate in the gastrointestinal tract and release the active pharmaceutical ingredient contained in the drug for absorption into the patient's systemic bloodstream. Where the solid dosage product is designed to be absorbed into the systemic bloodstream in the gastrointestinal tract, the solid dosage form is administered in the form of a tablet or a powder. Where the solid dosage route is a tablet, the blended tablet components are compacted under high compressive pressure to prevent the tablet from disintegrating before it reaches the gastrointestinal tract.

Enterocoated capsules are taken by individuals on a daily basis and are designed to deliver medications which are absorbed through the gastrointestinal system. However, such conventional delivery is not without its complications and may include gastric bleeding as well as degradation of the medicine by proteolytic enzymes in the gastrointestinal system.

However, for certain active pharmaceutical ingredients or for substances that exhibit therapeutic activity, absorption of the medication into the systemic bloodstream is required before the medication reaches the gastrointestinal tract to preclude degradation of the medication by the acidity and enzymes in the stomach. For such medications, delivery through the conventional tablet dosage form where the tablet is designed to disintegrate in the gastrointestinal tract is not efficacious. Therefore, such medications need to be delivered so that absorption of the active occurs before the active reaches the gastrointestinal tract.

The invention capsule is designed to provide a solution to overcome these problems by melting instantly in the mouth of the individual allowing the inside content to be absorbed directly through the mucosa immediately into the bloodstream without any gastric distress or degradation of the medication.

The prior art has shown various oral and buccal delivery systems for therapeutic agents and active ingredients. See U.S. Pat. Nos. 7,018,653; 6,488,953; 6,183,775; 5,827,525; 5,035,252 and U.S. Patent Publication No. 2008/0317850.

However, none of these prior art systems disclose a medicinal melting capsule made of a material that has the same melting point as the human body temperature and includes a therapeutic amount of medication for treatment of a desired medical condition wherein the medication has been manipulated and modified for optimal oral mucosal absorption, preferably sublingual absorption.

The present invention system of delivery has the following advantages over known conventional methods of administering medications;

1. Faster way to achieve therapeutic blood levels;
2. Bypassing the stomach prevents gastrointestinal complications;
3. Lower medication doses are used to reach the same blood levels delivered using conventional methods;
4. Drug administration via the sublingual method of the invention is possible in unconscious patients without complications;
5. Absorption of the delivered medication is more accurate and predictive and not dependent on stomach content and interference.

### SUMMARY OF THE INVENTION

In the present invention, these purposes, as well as others which will be apparent, are achieved by providing a medicinal melting capsule comprised of a material that has the same melting point as the human body temperature and includes a therapeutic amount of medication for use in treatment of a desired medical condition; wherein the medication has been modified for oral mucosal absorption. Preferably delivery of the invention capsule is sublingual

The medicinal melting capsule is made of a material that is stable and does not react with the atmosphere or with the medication within the capsule wall. The material is selected from the group consisting of chocolate, marshmallow, gelatin, starch, polysaccharide, lipids, proteins and carbohydrates. The capsule is designed to melt almost instantaneously when placed in an individual's mouth, preferably under the tongue. This immediately delivers the medication immediately into the bloodstream for fast-acting treatment.

Optionally, the medicinal melting capsule may contain an outer layer to prevent the capsule from melting before use as well as an inner outer layer to prevent the capsule from melting before use. These additional layers are optional, as they may slightly increase the melting time of the capsule therefore slowing the absorption of the medication into the bloodstream.

The medication within the capsule has been modified by changing factors selected from the group consisting of solvent, pH, buffer material and polarity to increase oral mucosal absorption. The medication is delivered in either in liquid or powder form and does not interact or degrade within the capsule.

The invention also includes a method for delivering medication to an individual via oral mucosal absorption by providing a melting capsule made of a material that has the same melting point as the human body temperature, and includes a therapeutic amount of medication for use in treatment of a desired medical condition. The medication is modified by manipulating the solvent, pH, buffer material and/or polarity to adjust for optimal oral mucosal absorption. The capsule is administered orally in the patient such that the capsule melts at the individual's body temperature and delivers the medication directly into the bloodstream of the individual quickly and without gastric complications.

Other objects, features and advantages of the present invention will be apparent when the detailed description of the preferred embodiments of the invention is considered which should be construed in an illustrative and not limiting sense.

### DETAILED DESCRIPTION OF THE INVENTION

As used herein, the term “mucosal” absorption of a medication and the like is meant to encompass absorption across or through a mucous membrane. In particular, “oral mucosal” absorption of a medication includes delivery across any tissue of the mouth, pharynx, larynx, trachea, particularly including the sublingual, gingival and palatal mucosal tissues.

The term “oral mucosal” absorption as used herein refers to a dosage form wherein medication delivery occurs substantially via the mucosal route and not via swallowing followed by GI absorption. Such dosage forms are designed to provide for a dissolution rate that allows for maximal delivery via the oral mucosa, typically via placement of the dosage form in the sublingual location.

As used herein, “sublingual”, means literally “under the tongue” and refers to a method of administering substances via the mouth in such a way that the substances are rapidly absorbed via the blood vessels under the tongue rather than via the digestive tract. Absorption occurs via highly vascularized buccal mucosa and allows a substance more direct access to the blood circulation, providing for direct systemic administration independent of gastrointestinal influences

In accordance with the present invention a medicinal melting capsule is provided that permits mucosal delivery of modified medications that, preferably, is intended to be absorbed directly in the mouth of the individual. The medication provided in the capsule is in a liquid or powder form which is easily absorbed into the bloodstream.

The material of the melting capsule has the same melting point as the human body temperature and can maintain the modified medication that has been adjusted for oral absorption in a stable condition, thus, preventing reaction with the atmosphere, or, with the capsule wall itself. The active ingredient will be released ONLY when the capsule is melted inside the mouth when the desired temperature has been reached

The original medication is modified in order to enhance optimal sublingual absorption that otherwise would not have been possible due to variant reactions with the air light and humidity. Modifications to the original medication can include a change in the nature of the solvent using water, alcohol and other solvents. A change in solubility: from being fat soluble to water soluble, or vice versa. Different pH values of the absorption area which are achieved by addition of buffer material. Changes can be made in the molecule itself to form more or less polarity to enhance absorption.

Different flavors and artificial sweeteners can be added to suit individuals tastes and specific medical conditions, such as diabetic's needs.

Different materials are used to manufacture the invention capsules in order to achieve, on the one hand, maximum protection of the active ingredients and on the other hand to achieve very fast and complete absorption of the medication. In addition the preferred material chosen for such purposes is not harmful and on the contrary it may have benefits and added value.

The invention capsule is made from different melting lipids, carbohydrates and/or proteins including chocolate, marshmallow, gelatin, starch and polysaccharides. These materials have the same melting point as the human body temperature and therefore easily melt when administered orally. In the preferred embodiment the capsule material is chocolate, with or without sugar. Optionally, the outer layer of the chocolate capsule can be covered with a polysaccharides layer to protect the chocolate so as not to melt before use, in case the temperature went too high.

Although the capsule can be made of a variety of materials, mentioned above, chocolate is preferred. The capsule when made of a thin layer of chocolate keeps the active material maintained in it's optimal condition without interacting with the air light or capsule wall until the moment of usage. The capsule melts instantly when needed and is the optimal way to deliver liquid to the sublingual space. The said liquid is mixed with the chocolate forming an optimal mixture that can adhere to the palate in order to be completely absorbed. Other materials (not chocolate)if mixed with liquid can form inadequate mixture that can lead to catastrophic aspiration especially in an unconscious patient.

In general use of chocolate has many advantages: it is a strong anti oxidant; it reduces LDL and increases HDL; and it is also a strong anti platelet aggregation action and vasodilator. Use of dark chocolate is most preferred because of its exceptional medicinal qualities and is preferred with diabetic patients.

The use of a chocolate capsule provides two optimal physical properties:

1. The ability to contain liquid as a mini container without interacting or allowing any leakage to occur before clinical usage; and
2. A melting point identical to the body temperature allowing the sublingual melting capsule to melt when it is placed under the tongue.

In addition, the chocolate has a number of biochemical actions that put it in the first line of defense against coronary, cerebro- and peripheral vascular disease. Specifically, chocolate is:

A very potent anti platelet aggregation drug reducing notably the thrombaxane;

A strong antioxidant preventing LDL cholesterol to be oxidized;

Increases levels of HDL (good cholesterol) REF1; and
Protects endothelial cell function and increases prostacycline secretion.

The invention capsules are designed to include any and all medications that can be delivered in a liquid or powder form, thus providing for treatment of a wide variety of conditions.

The use of the invention sublingual melting capsule is particularly useful in chronic cases where conventional delivery of medications by swallowing would be anticipated to cause stomach irritation bleeding or perforating, i.e. aspirin.

The invention capsules are also useful in medical emergencies where therapeutic blood levels of the specific medication are urgently needed but there is no intravenous line available, such as during grand mal seizures which occur in and unexpected place and time. There is a direct relationship between the length of the convulsions and the resulting permanent or temporary brain damage. Administering the invention capsule containing diazepam, the medication typically used to treat grand mal seizures, causes fast absorption into the bloodstream and crosses the blood brain barrier to interrupt convulsions in seconds thus preventing brain damage that would have occurred if such a vehicle did not exist.

The concentration of the active ingredient of the proposed medication is adjusted to be absorbed instantly and completely through the mouth mucosa. Preferably the active ingredient is in the form of a solution, although they can also be in powder form as long as the molecular size is small and/or polar to enhance passive absorption. The active ingredients according to their chemical structure, fat and water solubility, optimal ph and physiological temperature will be dissolved in a solution such that it can be passively absorbed in the mouth to reach therapeutic levels in the blood and if necessary to cross the blood brain barrier.

Acetyl salicylic acid (aspirin) is known to induce gastrointestinal complications, part of these serious side effects are due to the fact that the aspirin being strong cox inhibitor inhibits prostaglandin formation thus reducing stomach lubrication leading to perforation and bleeding systemic effect), however the aspirin being a strong acid can cause local damage to the gastric mucosa. In an attempt to reduce the said gastric complications aspirin is dissolved and placed into the sublingual melting capsule to be absorbed under the tongue thus achieving complete bypass of the of the gastric mucosa.

In acute myocardial infarction cases when fast action of the aspirin is needed the sublingual melting capsule containing the dissolved aspirin is administrated under the tongue even in unconscious patients to achieve therapeutic blood levels in less than four minutes.

It is obvious that these kinds of patients cannot use the chewable aspirin due to their physical condition. Although use of chewable aspirin can reach optimal blood levels within minutes, such chronic use is also associated with very severe tooth and gum complications that do not occur with the invention mode of administration

Uncontrolled blood pressure can cause serious medical complications such as a stroke. Hypertensive crisis is a medical emergency that if not treated instantly will bring upon catastrophic results. It has been proven that uncontrolled blood pressure before coronary bypass surgery is the worse predictor (worse than poor left ventricle function or diabetes) for successful outcome. When the blood pressure is excessively high at a certain measurement intravenous administration of hypotensive drug carries a substantial risk of lowering the blood pressure too much leading to haemodynamic shock. Parenteral administration of hypotensive tablets will take half an hour to one hour to work (which is too risky under these circumstances) and the amount of the active ingredient absorbed is completely unpredictable. In this and similar clinical conditions, the invention sublingual melting capsule including nifidipen, would have been very beneficial to administer. Nifidipen in the invention capsule permits gradual and accurate control of blood levels. Nifidipin is a calcium channel blocker that is absorbed easily sublingually once the capsule has melted. It is absorbed in its powder form and is reaches optimal blood levels within two to four minutes.

The foregoing description of various and preferred embodiments of the present invention has been provided for purposes of illustration only, and it is understood that numerous modifications, variations and alterations may be made without departing from the scope and spirit of the invention as set forth in the following claims.

What is claimed is:

1. A medicinal melting capsule comprised of a material that has the same melting point as the human body temperature and includes a therapeutic amount of medication for use in treatment of a desired medical condition; wherein said medication has been modified for oral mucosal absorption.

2. The medicinal melting capsule according to claim 1, said medication has been modified for sublingual absorption.

3. The medicinal melting capsule according to claim 1, wherein said material is stable and does not react with the atmosphere or with said medication within the capsule wall.

4. The medicinal melting capsule according to claim 1, further containing an outer layer to prevent the capsule from melting before use.

5. The medicinal melting capsule according to claim 1, further containing an inner outer layer to prevent the capsule from melting before use.

6. The medicinal melting capsule according to claim 1, wherein said material is selected from the group consisting of chocolate, marshmallow, gelatin, starch, polysaccharide, lipids, proteins and carbohydrates.

7. The medicinal melting capsule according to claim 1, wherein said medication is in powder or liquid form.

8. The medicinal melting capsule according to claim 1, wherein said medication has been modified by changing factors selected from the group consisting of solvent, pH, buffer material and polarity to increase oral mucosal absorption.

9. A method for delivering medication to an individual via oral mucosal absorption comprising the steps of:
providing a melting capsule comprised of a material, that has the same melting point as the human body temperature, and includes a therapeutic amount of medication for use in treatment of a desired medical condition;
modifying said medication by manipulating the solvent, pH, buffer material and/or polarity to adjust for oral mucosal absorption;
administering the capsule sublingually in the patient such that the capsule melts at the individual's body temperature and delivers the medication directly into the bloodstream of the individual quickly and without gastric complications.

10. The method according to claim 9, wherein the medication is in a liquid or powder form.
